(19) United States
(12) Patent Application Publication
(10) Pub. No.: US 2008/0306257 A1
(43) Pub. Date: Dec. 11, 2008

(54) Syntheses and Preparations of Narwedine and Related Novel Compounds

(75) Inventors: Gabriel Tojo Suarez, Ames (A Coruna) (ES); Ernesto Duran Lopez, Barcelona (ES); Jordi Bosch I Llado, Girona (ES)

Correspondence Address: HOGAN & HARTSON LLP;IP GROUP, COLUMBIA SQUARE, 555 THIRTEENTH STREET, N.W., WASHINGTON, DC, 20004, US

(73) Assignee: MEDICHEM, S.A., Barcelona (ES)

(21) Appl. No.: 11/913,649

(22) PCT Filed: May 3, 2006
(86) PCT No.: PCT/IB06/02875

§ 371 Date: Aug. 8, 2008

Related U.S. Application Data
(60) Provisional application No. 60/676,964, filed on May 3, 2005.
(60) Provisional application No. 60/722,015, filed on Sep. 30, 2005.

(51) Int. Cl. C07D 491/06

(52) U.S. Cl. 540/521; 540/581

(57) ABSTRACT

The present invention relates to a process for preparing racemic narwedine (which can be can be kinetically resolved) to yield (−)-narwedine and which is the biogenic precursor of (−)-galanthamine) and the use thereof as a starting material for producing (−)-galanthamine. The invention further includes processes for preparing (−)-galanthamine and (−)-galanthamine hydrobromide, as well as related novel compounds.

### CROSS REFERENCE TO RELATED APPLICATIONS

This application claims priority to U.S. Provisional Application Nos. 60/676,964, filed May 3, 2005 and 60/722,015, filed Sep. 30, 2005. These applications are expressly incorporated herein by reference in their entirety.

### BACKGROUND OF THE INVENTION

1. Field of the Invention

The invention relates to a process for preparing racemic narwedine (which can be can be kinetically resolved to yield (−)-narwedine and which is the biogenic precursor of (−)-galanthamine) and the use thereof as a starting material for producing (−)-galanthamine. The invention further includes processes for preparing (−)-galanthamine and (−)-galantamine hydrobromide.

2. Discussion of the Related Art

(−)-Galanthamine, a tertiary alkaloid having acetylcholinesterase inhibitor properties, has been isolated from the bulbs of the Caucasian snowdrops Galanthus woronowii. It has also been isolated from the common snowdrop Galanthus nivalis.

(−)-Galanthamine has been marketed by Waldheim (Sanochemia Gruppe) as Nivalin® in Germany and Austria since the 1970s for indications such as facial neuralgia.

(−)-Galanthamine, and derivatives thereof, are useful for the treatment of Alzheimer's disease and related illnesses.

(−)-Galanthamine hydrobromide has been approved by the FDA for the treatment of Alzheimer's disease.

The chemical name of (−)-galanthamine is (4aS,6R,8aS)-4a,5,9,10,11,12-hexahydro-3-methoxy-11-methyl-6H-benzofuro[3a,3,2-ef][2]benzazepin-6-ol. Both the base compound and its hydrobromide are levorotatory.

The crystal structure (−)-galanthamine hydrobromide is described in Acta Cryst., C53, 1284-1286 (1997).

Currently, (−)-galanthamine is obtained by extraction from natural sources, such as daffodils or snowdrops. The yields of these extraction process are low, expensive for pharmaceutical grade material and limited supplies of naturally-obtained (−)-galanthamine. (−)-Narwedine is the biogenic precursor of (−)-galanthamine.

An alternate source of (−)-galanthamine is its chemical synthesis. Some synthetic routes to (−)-galanthamine and racemic galanthamine have been published including, for example, (i) Barton and Kirby, J. Chem. Soc., 806 (1962); (ii) Shimizu et at, Heterocycles, 8, 277 (1977); (iii) Shimizu et at, Chem. Pharm. Bull, 26, 3765 (1978); (iv) Szewczyk et at, J. Heterocycl. Chem., 25, 1809 (1988); (v) Kametani et at, J. Org. Chem., 36, 1295 (1971); (vi) Kametani et al, J. Chem. Soc. Perkin Trans. 1, 1513 (1972); (vii) Kametani et al, J. Heterocycl. Chem., 10, 35 (1973); (viii) Vlahov et al, Tetrahedron, 45, 3329 (1989) and (ix) Holton et al, J. Am. Chem. Soc., 110, 314 (1988).

In J. Chem. Soc. (C), 2602-2605 (1969), the oxidative ring coupling of 2-bromo-5-hydroxy-N-[2-(4-hydroxyphenyl)ethyl]-4-methoxy-N-methylbenzamide (Compound 4) to 1-bromo-12-oxo-narwedine (Compound 5) was first described as a key step in the synthesis of racemic narwedine. Notably, the oxidation is described as being performed in a mixture of chloroform and water (5:1, 300 volumes) as solvent, using 5.66 molar equivalents of potassium hexacyanoferrate (III) as oxidant and 11.32 molar equivalents of sodium bicarbonate as base, at 60° C. for 1.5 hours, with a 40% yield after purification by means of column chromatography.

WO 96/31458 describes the oxidative ring coupling of Compound 4 in a two-phase liquid system made up of an aqueous base and an organic solvent having a dielectric constant less than 4.8 (as measured at 20° C.). Example 2 of WO 96/31458 is carried out with Compound 4 as starting material and a mixture of toluene and water (2:1, 150 volumes) as solvent, using 3.00 molar equivalents of potassium hexacyanoferrate (III) as oxidant and 11.32 molar equivalents of sodium bicarbonate as base, at reflux temperature (87° C.) for 3 hours. The yield was 19.1%.

Example 34 of WO 96/12692 describes the oxidative ring coupling of Compound 4 in a mixture of toluene and water (15:1, 320 volumes) as solvent, using 520 molar equivalents of potassium hexacyanoferrate (III) as oxidant and 5.50 molar equivalents of potassium carbonate as base, at 60° C. for 35 minutes, with a 37.8% yield after chromatographic purification. Potassium carbonate is a dibasic salt and therefore the reaction was carried out using 11.0 molar equivalents of base.

Synth. Commun., 30, 2833-2846 (2000) describes the oxidative ring coupling of Compound 4 using phenyliodine(III)bis(trifluoroacetate) (PIFA) as oxidant in trifluoroethanol at −40° C. in a 60% yield after column chromatography. Notwithstanding the yield, the need for chromatographic purification, the high cost of both oxidant and solvent, and also the low temperatures needed make the process inadequate for an industrial production.

J. Chem. Soc. (C), 2602-2605 (1969) describes the reduction of 1-bromo-12-oxo-narwedine (Compound 5) using lithium aluminum hydride that yields a mixture of about 60:40of galanthamine and epigalanthamine, where both are in their racemic forms. This mixture is then further purified by means of column chromatography to yield both products in their racemic forms.

J. Heterocyclic Chem., 32, 195-199 (1995) describes a method for resolving racemic galanthamine that involves derivatizing galanthamine using (−)-camphanic acid chloride and separating the diastereomeric mixture using column chromatography. The (+)-galanthamine present in the racemic mixture is of little or no commercial value and it can not be transformed into the desired enantiomer.

In this regard, U.S. Pat. No. 6,043,359 describes the preparation of narwedine from Compound 5 above (see examples 35 and 36) in which the carbonyl of Compound 5 is protected by using propyleneglycol to form a ketal (Scheme 1). The yield of this protection step is low (˜30.1%). Thereafter, the amido group and bromoaryl functionality are reduced with LiAlH₄, and the carbonyl is deprotected to yield narwedine. The yield of these last two steps is 61.2%, and the overall yield of converting Compound 5 to narwedine is only 18.4%

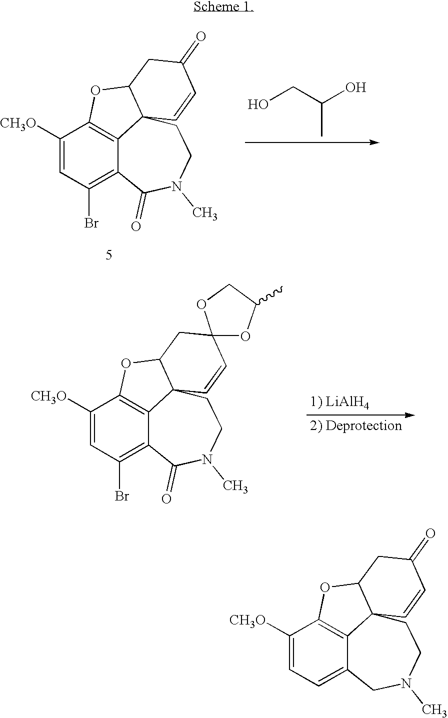

U.S. Pat. No. 5,428,159 (the “'159 patent”) describes the reduction of (−)-narwedine to (−)-galanthamine. According to Example VIII of the '159 patent, (−)-narwedine is reduced at −78° C. with L-selectride in THF to (−)-galanthamine. The mixture solution is stirred at −78° C. for 2 hours and then warmed to 0° C. Methanol is added, and the solution is warmed to 25° C. and stirred for 15 minutes. Solvent is then evaporated under vacuum to dryness to obtain a syrup. The syrup is dissolved in chloroform and the solution is loaded into a SiCh column. The column is eluted with a solvent mixture of chloroform and methanol (6/1) to obtain 286 mg of pure (−)-galanthamine (a yield of 99.5%). HPLC of the resultant mixture indicates there is no epigalanthamine or lycoramine present (m.p. 128° C.-129° C.; [α]_{D}²⁵=−93.4). Natural (−)-galanthamine has a melting point of 126-127° C. ([α]_{D}²⁵= 91.0).

Reactions at −78° C., however, are not very suitable to large scale production. Furthermore, the desired product must be purified by column chromatography, which is not affordable at industrial scale.

Org. Process Res. Dev., 3, 425-431 (1999) discloses that solutions of (−)-narwedine in tetrahydrofuran undergo racemization, and because of that, solid (−)-narwedine is portionwise added through a solid-addition funnel to solutions of L-selectride at temperatures below −15° C. Additionally, Org. Process Res. Dev., 3, 425-431 (1999) discloses that the final (−)-galanthamine is isolated after formation of its hydrobromic acid salt and further filtration. Although the disclosed process indicates mat the reduction reaction is carried out under an argon atmosphere, no information is provided regarding oxygen removal in subsequent steps and/or the purity of the final product.

Notwithstanding these advances, the synthetic approach to (−)-galanthamine production in high purity pharmaceutical grade on a commercial scale is still problematic.

### SUMMARY OF THE INVENTION

The invention relates to a process for preparing racemic narwedine (which can be kinetically resolved to yield (−)-narwedine and which is the biogenic precursor of (−)-galanthamine) and the use thereof as a starting material for producing (−)-galanthamine. The invention further includes processes for preparing (−)-galanthamine.

(−)-Galanthamine is obtained in substantially high yield and purity substantially without concomitant production of epigalanthamine, (+)-galanthamine or lycoramine by conversion of (−)-narwedine to (−)-narwedine-BF₃ complex and subsequent reduction to (−)-galanthamine-BF₃ complex using L-selectride. (−)-Galanthamine-BF₃ complex is then converted into the desired (−)-galanthamine.

Thus, the invention includes a process for the manufacture of enantiomeric (−)-galanthamine and/or its BF₃ complex comprising reducing enantiomeric (−)-narwedine-BF₃ complex. The (−)-narwedine-BF₃ complex is prepared by reaction of (−)-narwedine with BF₃.

The (−)-galanthamine hydrobromide obtained by the process of the invention exhibits a powder X-Ray diffraction pattern substantially identical to (−)-galanthamine hydrobromide present in the marketed Reminyl® and to the powder X-Ray diffraction pattern obtained by simulation from the single crystal structure data published in Acta Cryst., C53, 1284-1286 (1997).

The invention further includes a process for preparing racemic narwedine (which can be kinetically resolved to yield (−)-narwedine and which is the biogenic precursor of (−)-galanthamine) and the use thereof as a starting material for producing (−)-galanthamine. In particular, the invention provides an efficient process for the conversion, via oxidation, of racemic galanthamine, racemic epigalanthamine and/or mixtures thereof into racemic narwedine.

The invention further includes a process for the conversion of racemic galanthamine, racemic epigalanthamine and/or mixtures thereof into racemic narwedine and the further conversion thereof into (−)-narwedine.

The invention further includes a process for using the (−)-narwedine obtained according to the above processes for preparing (−)-galanthamine.

The invention further includes a process for preparing (−)-galanthamine hydrobromide by conversion of (−)-narwedine to (−)-narwedine-BF₃ complex, subsequent reduction to (−)-galanthamine-BF₃ complex using L-selectride as reducing agent and then the conversion of the same into (−)-galanthamine with a low amount of dehydration of galanthamine impurity (dehydroxygalanthamine).

The invention further includes processes having reaction conditions that improve significantly the yield of the oxidative ring coupling of 2-bromo-5-hydroxy-N-[2-(4-hydroxyphenyl)ethyl]-4-methoxy-N-methylbenzamide to produce 1-bromo-12-oxo-narwedine, which is a key intermediate in some synthetic routes to racemic narwedine.

The invention further includes a process for reducing 1-bromo-12-oxo-narwedine into racemic galanthamine, racemic epigalanthamine and/or mixtures thereof having a low content of undesired by-products. The obtained mixtures are useful as starting materials for producing narwedine having a low content of the undesired by-products and which is useful for producing (−)-galanthamine having a low content of the undesired by-products.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 illustrates the IR spectrum of (−)-narwedine;

FIG. 2 illustrates the IR spectrum of (−)-narwedine-BF₃ complex;

FIG. 3 illustrates the IR spectrum of (−)-galanthamine;

FIG. 4 illustrates the IR spectrum of (−)-galanthamine-BF₃ complex;

FIG. 5 illustrates the X-ray powder diffractogram of (−)-galanthamine hydrobromide;

FIG. 6 illustrates the X-ray powder diffractogram of (−)-narwedine; and

FIG. 7 illustrates the X-ray powder diffractogram of (−)-narwedine-BF₃ complex.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein. In addition, and as will be appreciated by one of skill in the art, the invention may be embodied as a method, system or process.

The invention relates to a process for preparing racemic narwedine (which can be kinetically resolved to yield (−)-narwedine and which is the biogenic precursor of (−)-galanthamine) and the use thereof as a starting material for producing (−)-galanthamine. The invention further includes processes for preparing (−)-galanthamine.

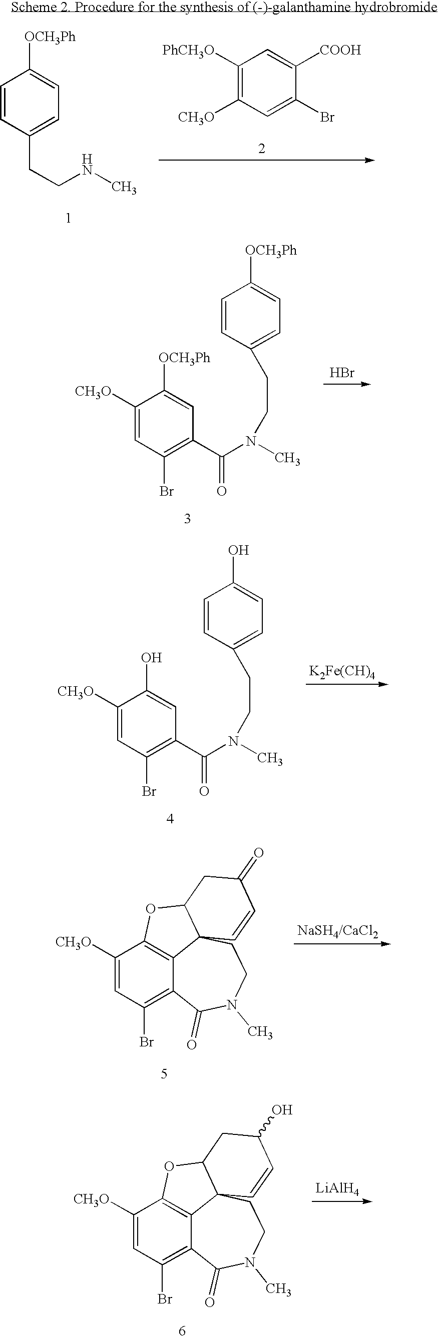

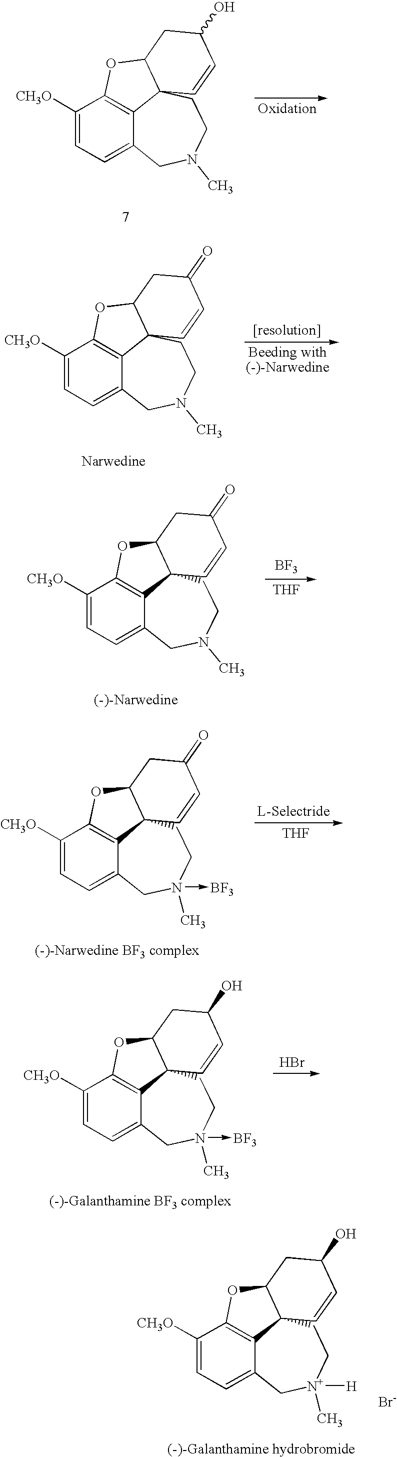

In particular, the invention relates to the use of a limited amount of base in the oxidative ring coupling of 2-bromo-5-hydroxy-N-[2-(4-hydroxyphenyl)ethyl]-4-methoxy-N-methylbenzamide (Compound 4) to 1-bromo-12-oxo-narwedine (Compound 5). This process provides a high yield in the transformation when, for example, potassium hexacyanoferrate (III) is used as the oxidant and without the need of chromatographic purification, thus making the process suitable for an industrial production.

It has been further observed that in this process, the addition of more than 10 molar equivalents of base has a detrimental effect on the yield. Therefore, the oxidative ring coupling reaction of the invention is performed using from approximately 0 to approximately 10 molar equivalents of base, preferably from approximately 2 to approximately 8 molar equivalents of base, and more preferably approximately 5 molar equivalents of base.

It has been further observed that the use of a mild base, such as sodium bicarbonate or potassium bicarbonate, instead of potassium carbonate, avoids the decomposition of the reaction mass under the reaction conditions thus leading to higher reaction yields. It has been further observed that the use of organic amines, such as triethylamine, diisopropylethylamine, and NH₃ are also suitable for the oxidative ring coupling reaction.

It has been further observed that improved yields of high purity 1-bromo-12-oxo-narwedine can be isolated if the reaction is carried out at lower temperatures than those described in the known processes using potassium hexacyanoferrate (III) as oxidant (i.e., higher than 60°Q.

The oxidative ring coupling reaction of the invention is typically carried out at a temperature between approximately 0° C. and approximately 50° C., preferably between approximately 15° C. and approximately 35° C., and more preferably between approximately 20° C. and approximately 25° C.

The invention further includes performing the oxidative ring coupling reaction of the invention using particular solvents. Namely, it has been observed that the oxidative ring coupling conditions (equivalents of base and reaction temperature) are not limited by the dielectric constant of the solvent (as measured at 20° Q. Examples of suitable solvents include toluene (dielectric constant 2.4), xylene (dielectric constant 2.4), methyl tert-butyl ether (dielectric constant 4.5), chloroform (dielectric constant 4.8), w-butyl acetate (dielectric constant 5.1), tert-butyl acetate (dielectric constant not available), isopropylacetate (dielectric constant not available), ethyl acetate (dielectric constant 6.0), tetrahydrofuran (dielectric constant 7.6), dichloromethane (dielectric constant 9.1) and acetonitrile (dielectric constant 37.5). The most preferred solvents are dichloromethane, ethyl acetate and tert-butyl acetate.

The invention also relates to a new process for preparing racemic narwedine (which can be kinetically resolved to yield (−)-narwedine and the use thereof as a starting material for producing (−)-galanthamine. In particular, the invention provides an efficient process for the conversion, via oxidation, of racemic galanthamine, racemic epigalanthamine and/or mixtures thereof into racemic narwedine that includes the steps of:
i. reducing 1-bromo-12-oxonarwedine (Compound 5); and
ii. oxidizing the product obtained in step i.

It has also been observed that an appreciable amount of lycoramine is obtained as an impurity during the reduction of 1-bromo-12-oxo-narwedine (Compound 5) when using lithium aluminum hydride due to the 1,4 reduction of the unsaturated ketone prior to the ketone reduction.

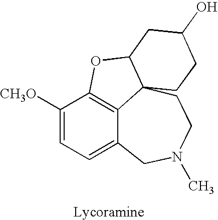

It has been further observed that an important amount of lycoramine is also obtained as impurity in the reduction of 1-bromo-12-oxonarwedine (Compound 5) if a borohydride salt is used for the reduction of the unsaturated ketone prior to the addition of lithium aluminum hydride to achieve the reduction of the amide and the bromoaryl functionalities of Compound 6.

The invention further includes die addition of a Lewis acid prior to the addition of the borohydride salt in the reduction of the unsaturated ketone of 1-bromo-12-oxo-narwedine that results in a decrease in the amount of 1-bromo-12-oxo-lycoramine as an impurity in 1-bromo-4a,5,9,10-tetrahydro-6-hydroxy-3-methoxy-11-methyl-6H-benzofuro[3a,32-ef][2]-benzazepin-12(9H)-one (Compound 6).

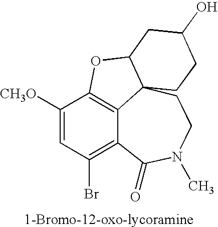

The invention further includes the use of 1-bromo-4a,5,9,10-tetrahydro-6-hydroxy-3-methoxy-11-methyl-6H-benzofuro[3a,3,2-ef][12]benzazepin-(Compound 6) with a low content of 1-bromo-12-oxo-lycoramine as impurity for an important decrease in the amount of lycoramine as impurity in the mixture of galanthamine and epigalanthamine (Compound 7).

The invention further includes the addition of a Lewis acid prior to the addition of the borohydride salt in the reduction of 1-bromo-12-oxonarwedine for the synthesis of narwedine with a very low amount of lycoraminone as impurity when the reduced mixture of galanthamine and epigalanthamine (Compound 7) is oxidized to narwedine.

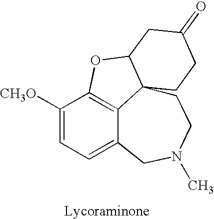

The invention further includes the addition of a Lewis acid prior to the addition of the borohydride salt in the reduction of 1-bromo-12-oxo-narwedine for the synthesis of (−)-galanthamine with a very low amount of lycoramine as impurity.

The invention further includes the use of 1-bromo-4a,5,9,10-tetrahydro-6-hydroxy-3-methoxy-11-methyl-6H-benzofuro[3a,32-ef][2]-benzazepin-12(9H)-one (Compound 6) with a low content of 1-bromo-12-oxo-lycoramine as impurity for the synthesis of final (−)-galanthamine with a very low amount of lycoramine as impurity.

Suitable Lewis acids for use in the above-described processes include cerium (III) chloride, cerium (III) bromide, lanthanum (III) chloride, iron (III) chloride, aluminum chloride, magnesium chloride, calcium chloride, lithium chloride, zinc chloride, nickel chloride, borane and trifluoroborane. Preferable Lewis Acids include cerium (III) chloride or calcium chloride, and more preferably cerium (III) chloride. The foregoing Lewis acids can be also be used in form of their hydrates or other solvates (e.g., cerium (III) chloride heptahydrate can be used as Lewis acid in the reduction of 1-bromo-12-oxo-narwedine with borohydride salts).

Suitable borohydride salts for use in the above-described processes include sodium borohydride, lithium borohydride, potassium borohydride, cesium borohydride, calcium borohydride, zinc borohydride, cerium borohydride, benzyltriethylammonium borohydride, tetramethylammonium borohydride, tetraethylammonium borohydride, tetrabutylammonium borohydride, cetyltrimethylammonium borohydride and methyltrioctylammonium borohydride. Preferred borohydride salts include sodium borohydride, lithium borohydride and potassium borohydride, and more preferably sodium borohydride.

Suitable solvents for use in the above-described reduction processes using borohydride salts in the presence of a Lewis acid include alcohols, aromatic solvents, ethers, dichloromethane, chloroform, alkenes, water and mixtures thereof. Preferred solvents include alcohols, and more preferably methanol. Pure solvents or mixtures thereof can be used in the reduction processes using borohydride salts in the presence of a Lewis acid (e.g., a mixture of toluene, methanol and isopropanol can be used as solvent in the reduction of 1-bromo-12-oxo-narwedine with borohydride salts in the presence of a Lewis acid).

The reduction processes using borohydride salts in the presence of a Lewis acid can be performed at temperatures ranging between the melting point and the boiling point of the solvent(s) and/or solvent mixtures, preferably between approximately −20° C. to approximately 30° C., and more preferably at approximately 0° C.

In the above described processes, the 1-bromo-4a,5,9,10-tetrahydro-6-hydroxy-3-methoxy-11-methyl-6H-benzofuro[3a,3,2-ef][2]benzazepin-12(9H-one (Compound 6) obtained in the first step of the process can be isolated or used directly in the subsequent reduction step without further isolation provided the solvent mixture is compatible with the use of lithium aluminum hydride.

In the above described processes, the amount of 1-bromo-12-oxo-lycoramine as an impurity in 1-bromo-4a,5,9,10-tetrahydro-6-hydroxy-3-methoxy-11-methyl-6H-benzofuro[3a,3,2-ef][2]-benzazepin-12(9H)-one (Compound 6) obtained by conjugated reduction with borohydride salts in the presence of a Lewis acid is approximately 0.0 to approximately 5.0% by HPLC area, preferably approximately 0.0 to approximately 1.0% by HPLC area, and more preferably approximately 0.0 to approximately 0.2% by HPLC area.

In the above described processes, the amount of lycoramine as an impurity in the galanthamine and epigalanthamine mixture (Compound 7) obtained by reduction of 1-bromo-4a,5,9,10-tetrahydro-6-hydroxy-3-methoxy-11-methyl-6H-benofuro[3a,3,2-ef][2]-benzazepin-12(9H)-one (Compound 6) having a low amount of 1-bromo-12-oxo-lycoramine as an impurity is approximately 0.0 to approximately 5.0% by HPLC area, preferably approximately 0.0 to approximately 1.0% by HPLC area, and more preferably approximately 0.0 to approximately 0.2% by HPLC area.

The invention further includes a process for converting racemic galanthamine, racemic epigalanthamine and/or mixtures thereof into racemic narwedine and the further conversion thereof into (−)-narwedine that includes the steps of:
i. oxidizing racemic galanthamine, racemic epigalanthamine and/or mixtures thereof; and
ii. resolving the racemic narwedine obtained in step i.

The invention further provides a process for the conversion, via oxidation, of racemic galanthamine, racemic epigalanthamine and/or mixtures thereof into racemic narwedine. The racemic narwedine can be converted into (−)-narwedine and used to prepare (−)-galanthamine.

The oxidation of galanthamine to narwedine is described in the literature (see Chem. Ber., 95, 1348-1353 (1962), J. Chem. Soc., 806-817 (1962) and Chem. Pharm. Bull, 12, 696-705 (1964) where MnO₂ is used as oxidizing agent in CHCl₃; see also Eur. J. Med. Chem., 27, 673-687 (1992) where pyridinium chlorochromate (PCC) is used as oxidizing agent in CH₂Cl₂, and see also J. Org. Chem., 63, 4535-4538 (1998) and U.S. Pat. No. 3,364,219 where Swern-type oxidation is described). Notably, no example has been found of the oxidation of epigalanthamine to narwedine or of a mixture of galanthamine and epigalanthamine to narwedine.

In this regard, the mixture obtained when reducing Compound 6 using a metal reducing agent (e.g., lithium aluminum hydride) can then be oxidized to narwedine. The resulting narwedine is suitable for preparing (−)-galanthamine. Oxidation of racemic galanthamine, racemic epigalanthamine and/or mixtures thereof can be performed using a Swern or Oppenauer oxidation. The Oppenauer oxidation can be suitably performed using cyclohexanone as oxidizing agent with a high conversion of the starting material, which is not afforded using acetone under conventional Oppenauer conditions.

The invention also includes a process for preparing (−)-narwedine that includes the steps of:
i. reducing 1-bromo-12-oxo-narwedine (Compound 5);
ii. oxidizing the product obtained in step i; and
iii. resolving of the racemic narwedine obtained in step ii.

The invention further includes a process for using the (−)-narwedine obtained according to the above processes for preparing (−)-galanthamine.

The invention provides a process for preparing (−)-galanthamine in high yields and purity without the concomitant production of epigalanthamine, (+)-galanthamine or lycoramine by conversion of (−)-narwedine to (−)-narwedine-BF₃ complex and subsequent reduction to (−)-galanthamine-BF₃ complex using L-selectride. (−) Galanthamine-BF₃ complex is then converted into the desired (−)-galanthamine (Scheme 3).

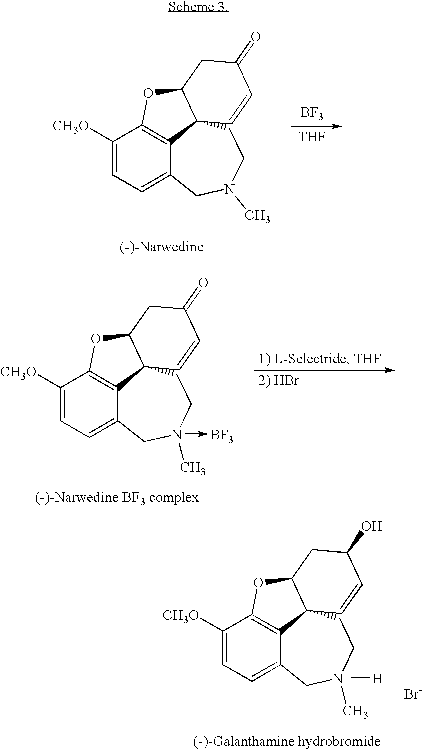

Thus, the invention includes a process for the manufacture of enantiomeric (−)-galanthamine and/or its BF₃ complex comprising reducing enantiomeric (−)-narwedine-BF₃ complex. The (−)-narwedine-BF₃ complex is prepared by reaction of (−)-narwedine with BF₃.

In this process, the (−)-narwedine-BF₃ complex is reduced, at suitable industrial temperatures for large scale production, using L-selectride to give high yields of highly pure (−)-galanthamine without substantial amounts of epigalanthamine or (+)-galanthamine or lycoramine. The reduction reaction is carried out at a temperature below room temperature, preferably between approximately −20° C. and approximately 0° C., and in an ether solvent, preferably a cyclic ether, more preferably tetrahydrofuran.

Advantage of this aspect of invention include, among others, the reduction reaction of (−)-narwedine-BF₃ complex can be carried out at approximately 0° C. Additionally, (−)-narwedine.BF₃ complex racemizes very slowly in THF solution at approximately 0° C.

During the reduction reaction, the solid is charged in the reactor before the addition of the solvent, which is the usual procedure at industrial scale. In the known processes, the solid (−)-narwedine is added over a cooled solution of a water-sensitive reagent (L-selectride at −15° C.). This addition needs special equipments such as solid-addition funnels.

It has been further observed that the reduction of (−)-narwedine to (−)-galanthamine using L-selectride proceeds with the formation of some amount of dehydroxygalanthamine as an impurity. The reduction of the (−)-narwedine-BF₃ complex with L-selectride also proceeds with the formation of some amount of dehydroxygalanthamine as impurity.

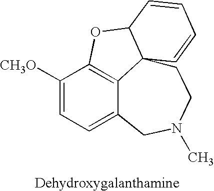

It has been further observed, and the invention includes, a process by which the amount of dehydroxygalanthamine produced as an impurity is dramatically reduced by (i) performing the reduction reaction under inert (e.g., nitrogen) atmosphere, (ii) bubbling all of the solutions and solvents with nitrogen to remove traces of dissolved oxygen prior to their addition to the reaction and, optionally, (iii) performing the post-reaction work-up under an inert atmosphere.

The use of L-selectride as a reducing agent yields tri-sec-butylborane which can undergo a very exothermic oxidation reaction to tri-sec-butylborate. It has been observed that hydrolysis of the tri-sec-butylborate is related with an increase in the amount of dehydroxygalanthamine produced as impurity in final (−)-galanthamine. Although not wishing to be bound by a particular theory, it is believed that the increase in the presence of the dehydroxygalanthamine impurity is due to (−)-galanthamine being dehydrated by these trialkylborates notwithstanding the presence of water in the reaction medium. Thus, the invention further includes a process by which the amount of dehydroxygalanthamine produced as an impurity is minimized by filtering a suspension of (−)-galanthamine hydrobromide in a solution of tri-sec-butylborane in THF under nitrogen atmosphere.

It has been further observed that the amount of dehydroxygalanthamine is dramatically reduced if the filtration of (−)-galanthamine hydrobromide, in suspension in a solution of tri-sec-butylborane in THF, is carried out under nitrogen atmosphere as the oxidation of it to tri-sec-butylborate is avoided.

It has been further observed that dehydration of (−)-galanthamine occurs if any excess of L-selectride is quenched prior to, or at the same time, that hydrobromic acid is added to form (−)-galanthamine hydrobromide. Thus, the invention further includes a process by which the amount of dehydroxygalanthamine produced as an impurity is minimized by quenching any excess L selectride under neutral or basic conditions prior to the addition of aqueous hydrogen bromide solution.

The invention further includes (−)-galanthamine hydrobromide produced by the above-described processes in which the amount of dehydroxygalanthamine therein is between approximately 0.00 and approximately 0.20% by HPLC area, preferably approximately 0.00 to approximately 0.10% by HPLC area, and more preferably approximately 0.00 and approximately 0.05% by HPLC area.

Optionally, further crystallization or purification steps known within the art can be utilized to reduce or remove any remaining epigalanthamine, (+)-galanthamine, lycoramine and/or dehydroxygalanthamine.

Notably, in contrast to known processes, the process of the invention results in significantly lower amounts of epigalanthamine, (+)-galanthamine and/or lycoramine being produced; in fact, substantially no epigalanthamine, (+)-galanthamine or lycoramine is produced by the processes of the invention.

It has been further observed that (−)-galanthamine hydrobromide can be further purified with high yield and without degradation of the material by crystallization in water in the presence of ammonium bromide. Lower yields are obtained using water in the absence of ammonium bromide due to the high solubility of (−)-galanthamine hydrobromide in water (about 50 mg/mL at room temperature and 300 mg/mL at reflux temperature). Yields could also be increased in the presence of hydrobromic acid, but (−)-galanthamine is rapidly epimerized to (−)-epigalanthamine under acidic conditions.

It has been further observed that the use of water as solvent in the final recrystallization of (−)-galanthamine hydrobromide avoids the presence of residual organic solvents in the active pharmaceutical ingredient (“API”). For example, (−)-galanthamine hydrobromide obtained from an ethanol-tetrahydrofuran mixture retains high amounts of both ethanol and tetrahydrofuran both which are not easily removed by conventional drying.

Additionally, crystallization of (−)-galanthamine hydrobromide in water in the presence of ammonium bromide afford high particle-size API that can be easily transformed into any required particle size distribution by simple milling.

The (−)-galanthamine hydrobromide obtained by the process of the invention exhibits a powder X-Ray diffraction pattern substantially identical to (−)-galanthamine hydrobromide present in the marketed Reminyl® and to the powder X-Ray diffraction pattern obtained by simulation from the single crystal structure data published in Acta Cryst., C53, 1284-1286 (1997).

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents.

### Specific Examples

The following examples are for illustrative purposes only and are not intended, nor should they be interpreted to, limit the scope of the invention.

### General Experimental Conditions:

i. HPLC Method 1:

Chromatographic separation was carried out using a Phenomenex Luna C18, 5 μm, 4.6×150 mm I.D column at room temperature (˜20-25° C.).

The mobile phase A was 0.01 M ammonium bicarbonate buffer (pH=7.5) which was prepared from 0.79 g of NH₄HCO₃ dissolved in 1000 mL of water. The pH was adjusted to 7.5 with formic acid. The mobile phase was mixed and filtered through a 0.22 μm nylon filter under vacuum.

The mobile phase B was acetonitrile.

The chromatograph was programmed as follows: 0-20 minutes isocratic 85% mobile phase A, 20-25 minutes linear gradient to 75% mobile phase A, 25-45 minutes isocratic 75% mobile phase A, 45-47 minutes linear gradient to 85% mobile phase A and 47-60 minutes equilibration to 85% mobile phase A.

The chromatograph was equipped with a 232 nm detector, and the flow rate was 1.0 mL per minute. Test samples (10 μL) were prepared by dissolving the appropriate amount of sample in order to obtain 1.0 mg per mL of a 85:15 mixture of mobile phases A and B.

ii. HPLC Method 2 (Chiral):

Chromatographic separation was carried out using a Daicel Chiralcel OD-H, 5 μm, 4.6×150 mm ID column at room temperature (˜20-25° C.).

The mobile phase A was hexane.

The mobile phase B was ethanol.

The chromatograph was programmed as follows: Initial 95% mobile phase A, 0-15 minutes linear gradient to 90% mobile phase A, 15-20 minutes isocratic 90% mobile phase A, 20-25 minutes linear gradient to 85% mobile phase A, 25-45 minutes isocratic 85% mobile phase A, 45-46 minutes linear gradient to 95% mobile phase A and 46-55 minutes equilibration to 95% mobile phase A.

The chromatograph was equipped with a 218 nm detector, and the flow rate was 1.0 mL per minute. Test samples (10 μL) were prepared by dissolving the appropriate amount of sample in order to obtain 1.0 mg per mL of a 50:50 mixture of hexane and sodium ethoxide solution (0.25 mg/ml) in ethanol.

iii. HPLC Method 3

Chromatographic separation was carried out using a Phenomenex Luna C18, 5 μm, 4.6×150 mm I.D column at room temperature (20-25° C.).

The mobile phase A was 0.01 M ammonium bicarbonate buffer (pH=7.5) which was prepared from 0.79 g of NH₄HCO₃ dissolved in 1000 mL of water. The pH was adjusted to 7.5 with formic acid. The mobile phase was mixed and filtered through a 0.22 μm nylon filter under vacuum.

The mobile phase B was acetonitrile.

The chromatograph was programmed as follows: Initial 0-20 minutes isocratic 85% mobile phase A, 20-25 minutes linear gradient to 75% mobile phase A, 25-45 minutes isocratic 75% mobile phase A, 45-47 minutes linear gradient to 85% mobile phase A and 47-60 minutes equilibration to 85% mobile phase A.

The chromatograph was equipped with a 232 nm detector, and the flow rate was 1.0 mL per minute. Test samples (10 μL) were prepared by dissolving the appropriate amount of sample in order to obtain 1.0 mg per mL of a 85:15 mixture of mobile phases A and B.

iv. HPLC Method 4

Chromatographic separation was carried out using a Waters Symmetry C18, 5 μm, 4.6×150 mm I.D column at room temperature (20-25° C.).

The mobile phase A was a mixture of 0.065 M ammonium acetate buffer (pH=6.5), which was prepared from 5.01 g of CH₃COONH₄ dissolved in 1000 ml of water. The pH was adjusted to 6.5 with acetic acid, and acetonitrile (65:35, v/v). The mobile phase was mixed and filtered through a 0.22 μm nylon filter under vacuum.

The mobile phase B was acetonitrile.

The chromatograph was programmed as follows: Initial 0-15 minutes isocratic 100% mobile phase A, 15-20 minutes linear gradient to 45% mobile phase A, 20-50 minutes isocratic 45% mobile phase A, 50-55 minutes linear gradient to 100% mobile phase A and 55-70 minutes equilibration to 100% mobile phase A.

The chromatograph was equipped with a 230 nm detector, and the flow rate was 1.0 mL per minute. Test samples (10 μL) were prepared by dissolving the appropriate amount of sample in order to obtain 1.0 mg per mL of mobile phase A for 2-bromo-5-hydroxy-N-[2-(4-hydroxyphenyl)ethyl]-4-methoxy-N-methylbenzamide or mobile phase B for 5-benzyloxy-N-[2-(4-benzyloxyphenyl)ethyl]-2-bromo-4-methoxy-N-methylbenzamide.

v. HPLC Method 5

Chromatographic separation was carried out using a Waters Symmetry C18, 5 μm, 4.6×150 mm I.D column at room temperature (˜20-25° C.).

The mobile phase A was water.

The mobile phase B was acetonitrile.

The chromatograph was programmed as follows: Initial 0-10 minutes isocratic 70% mobile phase A, 10-18 minutes linear gradient to 30% mobile phase A, 18-30 minutes isocratic 30% mobile phase A, 30-35 minutes linear gradient to 70% mobile phase A and 35-40 minutes equilibration to 70% mobile phase A.

The chromatograph was equipped with a 225 nm detector, and the flow rate was 1.0 mL per minute. Test samples (10 μL) were prepared by dissolving the appropriate amount of sample in order to obtain 1.0 mg per mL of acetonitrile.

vi. HPLC Method 6

Chromatographic separation was carried out using a Phenomenex Luna C18, 5 μm, 4.6×150 mm I.D column at 25° C.

The mobile phase was 0.01 M ammonium bicarbonate buffer (pH=7.5) which was prepared from 0.79 g of NH₄HCO₃ dissolved in 1000 mL of water. The pH was adjusted to 7.5 with formic acid and acetonitrile (85:15, v/v). The mobile phase was mixed and filtered through a 0.22 μm nylon filter under vacuum.

The chromatograph was equipped with a 232 nm detector, and the flow rate was 1.5 mL per minute. Test samples (10 μL) were prepared by dissolving the appropriate amount of sample in order to obtain 1.0 mg per mL of mobile phase.

vii. HPLC Method 7

Chromatographic separation was carried out using a Phenomenex Luna C18, 5 μm, 4.6×150 mm I.D column at room temperature (20-25° C.).

The mobile phase A was 0.01 M ammonium bicarbonate buffer (pH=7.5) which was prepared from 0.79 g of NH₄HCO₃ dissolved in 1000 mL of water. The pH was adjusted to 7.5 with formic acid. The mobile phase was mixed and filtered through a 0.22 μm nylon filter under vacuum.

The mobile phase B was acetonitrile.

The chromatograph was programmed as follows: Initial 0-20 minutes isocratic 85% mobile phase A, 20-25 minutes linear gradient to 75% mobile phase A, 25-65 minutes isocratic 75% mobile phase A, 65-67 minutes linear gradient to 85% mobile phase A and 67-80 minutes equilibration to 85% mobile phase A.

The chromatograph was equipped with a 232 nm detector, and the flow rate was 1.0 mL per minute. Test samples (10 μL) were prepared by dissolving the appropriate amount of sample in order to obtain 1.0 mg per mL of a 85:15 mixture of mobile phases A and B.

viii. HPLC Method 8 (Chiral)

Chromatographic separation was carried out using a Daicel Chiralcel OD-H, 5 μm, 4.6×150 mm I.D. column at room temperature (˜20-25° C.).

The mobile phase was hexane/ethanol (1% acetic acid) (50:50, v/v).

The chromatograph was equipped with a 230 nm detector, and the flow rate was 1.0 mL per minute. Test samples (40 μL) were freshly prepared by dissolving the appropriate amount of sample in order to obtain 0.25 mg per ml of mobile phase.

### Example 1

### Synthesis of 5-Benzyloxy-N-[2-(4-benzyloxyphenyl)ethyl]-2-brom bromo-4-methoxy-N-methylbenzamide (Compound 3)

A solution of 48.7 g of 5-benzyloxy-2-bromo-4-methoxybenzoic acid (Compound 2) in 1.08 L of toluene was heated to reflux, and residual water was removed azeotropically. The solution was cooled to 68° C., and 4.8 mL of N,N-dimethylformamide was added to the resulting suspension, followed by the addition of 12.6 mL of thionyl chloride. The suspension was then stirred at this temperature for two hours. Next, 350 mL of toluene was distilled at reflux temperature to remove excess of thionyl chloride, and the solution was cooled to room temperature. At this temperature, 24 mL of triethylamine was added to the solution. Thereafter, an anhydrous solution of 34.7 g of 2-(4-benzyloxyphenyl)ethylmethylamine (Compound 1) in 140 mL of toluene was added to the reaction mixture over 30 minutes. The resulting solution was stirred at room temperature for 1 hour. The solution was then washed with 200 mL of 5% NaHCO₃ solution, followed by 200 mL of 0.1 M HCl solution, and finally with 200 mL of water.

The organic phase was extracted and concentrated to a final volume of 160 mL. Next, 240 mL of isopropanol was added to the solution, and 80 mL of the resulting mixture of solvents was distilled at atmospheric pressure. After cooling to 40° C., the resulting solution was seeded with 5-benzyloxy-N-[2-(4-benzyloxyphenyl)ethyl]-2-bromo-4-methoxy-N-methylbenzamide (Compound 3). Next, the suspension was cooled at room temperature overnight and finally at 0° C. for 90 minutes. A pale beige solid was filtered using a Büchner funnel and washed with 160 mL of chilled isopropanol to yield 60.3 g of 5-benzyloxy-N-[2-(4-benzyloxyphenyl)ethyl]-2-bromo-4-methoxy-N-methylbenzamide (Compound 3) (Yield: 74.7%; HPLC (method 4) purity 94.5%).

### Example 2

### Synthesis of 2-Bromo-5-hydroxy-N-[2-(4-hydroxyphenyl)ethyl]-4-methoxy-N-methylbenzamide (Compound 4)

5-Benzyloxy-N-[2-(4-benzyloxyphenyl)ethyl]-2-bromo-4-methoxy-N-methylbenzamide (5.0 g) (Compound 3) was dissolved in a mixture of toluene (50 mL) and 48% hydrobromic acid (71 mL). The resulting solution was stirred at 60° C. for 2 hours. After cooling to room temperature, the aqueous phase was extracted. CH₂Cl₂ (25 mL) was added over the aqueous phase, and the resulting mixture was cooled to 0° C. At this temperature, 120 mL of 20% aqueous NaOH solution was slowly added while maintaining the reaction temperature below 5° C. Precipitation of a white solid was observed during the addition. The pH was adjusted to 5 with NaHCO₃, and the solid was filtered using a Büchner funnel and washed with 6 mL of water and 6 mL of heptane to obtain a quantitative yield 2-Bromo-5-hydroxy-N-[2-(4-hydroxyphenyl)ethyl]-4-methoxy-N-methylbenzamide (Compound 4) (HPLC (method 4) purity 95.9%).

### Example 3

### Synthesis of 1-bromo-12-oxo-Narwedine (Compound 5) Using Dichloromethane as Solvent

In a 1 L, three necked round bottom flask equipped with mechanical stirring and N₂ inlet, 2.0 g (5.3 mmol) of 2-bromo-5-hydroxy-N-[2-(4-hydroxyphenyl)ethyl]-4-methoxy-N-methylbenzamide (Compound 4) was dissolved in 600 mL of CH₂Cl₂. To the resulting solution H₂O (40 mL), KHCO₃ (2.9 g, 29 mmol) and K₃[Fe(CN)₆] (8.4 g, 26 mmol) were added, and the resulting mixture was stirred at room temperature (˜20-25° C.) for 24 hours. The resulting mixture was filtered using a Büchner funnel and the mother liquors were decanted. The organic layer was evaporated to dryness to yield 1.4 g of 1-bromo-12-oxo-narwedine (Compound 5) (Yield: 71%; HPLC (method 5) purity 87%).

### Example 4

### Synthesis of 1-Bromo-12-oxo-Narwedine (Compound 5) Using Toluene as Solvent

In a 1 L, three necked round bottom flask equipped with mechanical stirring and N₂ inlet, 2.0 g (5.3 mmol) of 2-bromo-5-hydroxy-N-[2-(4-hydroxyphenyl)ethyl]-4-methoxy-N-methylbenzamide (Compound 4) was dissolved in 600 mL of toluene. To the resulting solution H₂O (40 mL), KHCO₃ (2.9 g, 29 mmol) and K₃[Fe(CN)₆] (8.4 g, 26 mmol) were added, and the resulting mixture was stirred at room temperature (˜20-22° C.) for 20 hours. The resulting mixture was filtered using a Büchner funnel and the mother liquors were decanted. The organic layer was evaporated to dryness to yield 1.22 g of 1-bromo-12-oxo-narwedine (Compound 5) (Yield: 61%; HPLC (method 5) purity 90%).

### Examples 5-12

Examples 5-12 illustrate the reaction results obtained when Example 4 is performed using different solvents.

| | | | | HPLC purity |
|---|---|---|---|---|
| Example | Solvent | Base | Yield | (method 5) |
| 5 | AcO^{t}Bu | KHCO₃ (5.5 eq) | 91% | 71% |
| 6 | AcOⁱPr | KHCO₃ (5.5 eq) | 91% | 35% |
| 7 | AcOEt | KHCO₃ (5.5 eq) | 67% | 79% |
| 8 | AcOⁿBu | KHCO₃ (5.5 eq) | 54% | 28% |
| 9 | Xylene | KHCO₃ (5.5 eq) | 16% | 94% |
| 10 | Methyl tert-butyl | KHCO₃ (5.5 eq) | 8% | 81% |
| | ether | | | |
| 11 | Chloroform | KHCO₃ (5.5 eq) | 52% | 70% |
| 12 | THF | KHCO₃ (5.5 eq) | 19% | 15% |

### Examples 13-23

Examples 13-23 illustrate the reaction results obtained when Example 4 is performed using a different base or a different amount of base.

| | | | | HPLC purity |
|---|---|---|---|---|
| Example | Solvent | Base | Yield | (method 5) |
| 13 | Toluene | KHCO₃ (5.5 eq) | 61% | 90% |
| 14 | Toluene | KHCO₃ (2.0 eq) | 28% | 67% |
| 15 | Toluene | KHCO₃ (1.1 eq) | 21% | 47% |
| 16 | Toluene | — | 0% | — |
| 17 | Toluene | KHCO₃ (11.0 eq) | 24% | 89% |
| 18 | Toluene | KHCO₃/ | 37% | 78% |
| | | LiCl (5.5 eq) | | |
| 19 | Toluene | Li₂CO₃ (5.5 eq) | 38% | 80% |
| 20 | Toluene | NEt₃ (5.5 eq) | 43% | 87% |
| 21 | Toluene | Diisopropyl- | Not isolated. | — |
| | | ethylamine | Detected by TLC | |
| | | (5.5 eq) | | |
| 22 | Toluene | Pyridine (5.5 eq) | 0% | — |
| 23 | Toluene | NH₃ (5.5 eq) | Not isolated. | — |
| | | | Detected by TLC | |

### Example 24

### Synthesis of 1-Bromo-4a,5,9,10-tetrahydro-6-hydroxy-3-methoxy-11-methyl-6H-benzofuro[3a,3,2-ef][2]-benzazepin-12(9H)-one (Compound 6) Substantially Free of 1-Bromo-12-oxo-lycoraminone

A suspension of 423 g of 1-bromo-12-oxo-narwedine (Compound 5) and 12.7 g of cerium (III) chloride heptahydrate in a mixture of toluene (110 mL) and methanol (210 mL) was cooled to 0° C., and stirred at this temperature for 30 minutes. At this temperature, a suspension of 4.4 g of sodium borohydride in 50 mL of isopropanol was added over 40 minutes to the reaction mixture while maintaining the reaction temperature below 5° C. and the resulting suspension was stirred at 0° C. for additional 3 hours. Next, 425 mL of water was added to the reaction mixture, and the pH was adjusted to between 4 and 8. The organic solvents were distilled under vacuum, and the resulting aqueous suspension was extracted twice with 1.1 L of ethyl acetate at 50° C. The organic solvent was then evaporated to dryness, and 1-bromo-4a,5,9,10-tetrahydro-6-hydroxy-3-methoxy-11-methyl-6H-benzofuro[3a,3,2-ef][2]-benzazepin-12(9H)-one (Compound 6) was isolated in quantitative yield (HPLC (method 6) purity 98.0%—addition of two diastereomers-; no 1-bromo-12-oxo-lycoraminone was detected).

### Example 25

### Reduction of 1-Bromo-12-oxo-narwedine (Compound 5)

A 63 L reactor that had been inertized with nitrogen was charged with 12 L of THF to which 1.9 Kg of LiAlH₄ solution (15% in THF) was then added. Thereafter, a solution of 300 g of 1-bromo-12-oxo-narwedine in 18 L of THF was slowly added to the reactor at room temperature over 2 hours. The mixture was then heated to reflux for 5 hours and then cooled to room temperature. Excess hydride was then quenched using a mixture of THF and water. Next, the aluminate was removed by filtration, and the organic phase was extracted and evaporated to dryness resulting in an oily product. Upon standing, the oily product crystallized spontaneously (193 g, 90% yield).

### Example 26

### Synthesis of a Mixture of Galanthamine and Epigalanthamine (Compound 7) Substantially Free of Lycoramine

A solution of 42.5 g of 1-bromo-4a,5,9,10-tetrahydro-6-hydroxy-3-methoxy-11-methyl-6H-benzofuro[3a,3,2-ef][2]-benzazepin-12(9H)-one (Compound 6) in 85 mL of toluene was distilled at reflux temperature to remove residual water azeotropically. After cooling to room temperature, 335 mL of 2.0 M lithium aluminum hydride solution in tetrahydrofuran was added to the reaction mixture over 30 minutes. Next, the resulting solution was stirred at reflux temperature for 2 hours. Thereafter, the solution was cooled to room temperature and 170 mL of a mixture of tetrahydrofuran and water (1:1) was slowly added to the reaction mixture to yield a white suspension. Next, 215 mL of water and 9.5 mL of 50% aqueous NaOH solution were added to the reaction mixture. The resulting suspension was then filtered using a Büchner funnel. The isolated solid was washed twice with 85 mL of tetrahydrofuran and discarded. The mother liquors were then extracted, and the organic phase was concentrated to dryness to yield a mixture of galanthamine and epigalanthamine in quantitative yield (HPLC (method 7) purity 83.6%-addition of two diastereomers-; 0.08% lycoramine).

### Example 27-30

Examples 27-30 illustrate the reduction of Compound 5 to Compound 7 using different reduction agents.

| | Reduction agent | | |
|---|---|---|---|
| | For the | For the reduction | Lycoramine in |
| | reduction of the | of the amide and | Compound 7 (% by |
| Ex. | unsaturated ketone | bromoaryl functionalities | HPLC, method 7) |
| 27 | LiAlH₄ | LiAlH₄ | 8.7% |
| 28 | NaBH₄ | LiAlH₄ | 19.9% |
| 29 | NaBH₄ + Ce(NO₃)₃ | LiAlH₄ | 0.12% |
| 30 | NaBH₄ + CaCl₂ | LiAlH₄ | 2.85% |

### Example 31

### Oxidation of the Galanthamine/Epigalanthamine Mixture

A solution of 37.8 mL of oxalyl chloride in 1.6 L of CH₂Cl₂ was cooled to below −60° C. under nitrogen. To the cooled solution, 64.8 mL of DMSO in 360 mL of CH₂Cl₂ was added over 30 minutes. At the same temperature, a solution of 90 g of a galanthamine-epigalanthamine mixture dissolved in 360 mL of CH₂Cl₂ was added over 30 minutes. The mixture was then stirred for 2 hours while maintaining the temperature below −60° C. Next, 540 mL of triethylamine was added to the solution, and the resulting mixture was heated to room temperature. Thereafter, 4.2 L of water was added to the solution, and the organic phase was extracted and evaporated to dryness to yield crude narwedine (Yield: 97%; HPLC (Method 3) purity 90%). Recrystallization of the crude narwedine yielded pure narwedine (Yield: 66%; HPLC (Method 3) purity 98%).

### Example 32

### Oxidation of (−)-Galanthamine to Narwedine (Oppenauer Conditions using Acetone)

To a 130 mL mixture (50:50) of acetone and toluene was added 5.0 g of (−)-galanthamine and 5.8 g of aluminium isopropoxide. The resulting mixture was heated to reflux for 7 hours, and then cooled to room temperature. To the solution was added 50 mL of water and 80 mL of 1 M sulfuric acid. After extracting the aqueous phase, 100 mL of CH₂Cl₂ was added, and the solution was basified to pH 11 with NaOH. The organic phase was then extracted, and the solvent was evaporated to dryness to yield 4.9 g of solid (22% conversion by NMR).

### Example 33

### Oxidation of (−)-Galanthamine to Narwedine (Oppenauer Conditions Using Cyclohexanone)

In a suitable reactor, 1.17 Kg of (−)-galanthamine was dissolved in 10 L of toluene. To the solution was added 1.0 Kg of aluminium isopropoxide and 3.6 L of cyclohexanone. The resulting mixture was then heated to reflux for 5 hours and cooled to room temperature. Thereafter, 10 L of water and 1.6 L of 35% HCl were added to the reaction mixture. After extracting the aqueous phase, 7.6 L of CH₂Cl₂ was added, and the solution was basified with NaOH. Next, the aluminate was removed by filtration, and the solvent was distilled while isopropanol was added to the mixture to yield narwedine as a white solid precipitated from the solution (Yield: 63%; HPLC (Method 3) purity 95.5%).

### Example 34

### Synthesis of Narwedine Substantially Free of Lycoraminone

A solution of 3.8 mL of oxalyl chloride in 160 mL of CH₂Cl₂ was cooled to −60° C. under nitrogen. To the cooled solution, 6.5 mL of DMSO in 36 mL of CH₂Cl₂ was added over 30 minutes. At the same temperature, a solution of 9.0 g of a galanthamine-epigalanthamine mixture (0.18% lycoramine by HPLC method 7) dissolved in 36 mL of CH₂Cl₂ was added over 30 minutes. The mixture was then stirred for 2 hours while maintaining the temperature below −60° C. Next, 54 mL of triethylamine was added to the solution, and the resulting mixture was heated to room temperature. Thereafter, 420 mL of water was added to the solution, and the organic phase was extracted and evaporated to dryness. The residue was suspended in isopropanol at 0° C., and the resulting solid was isolated by filtration to yield narwedine. (Yield 98%; HPLC (method 7) purity: 91.5%; 0.06% lycoraminone). Recrystallization of the narwedine in an ethanol/water/acetic acid/triethylamine mixture afforded pure narwedine (Yield: 50%; HPLC (method 7) purity 99.9%; 0.03% lycoraminone).

### Example 35

By reproducing example 34 using a starting mixture with a content of 7.47% of lycoramine, the final recrystallized narwedine had a content of 2.86% of lycoraminone.

### Example 36

### Preparation of (−)-Narwedine from Racemic Narwedine

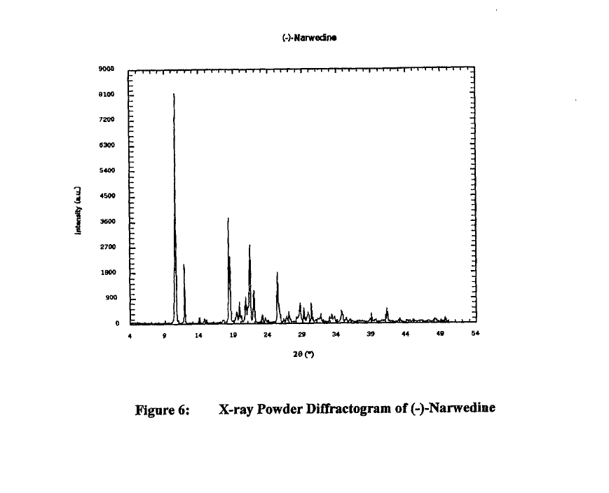

A suspension of 50.0 g of racemic narwedine in a mixture of 850 mL of ethanol, 50 mL of water and 12 mL of glacial acetic acid was heated to reflux temperature. After cooling to 75-80° C., 100 mL of triethylamine was added to the resulting solution. Next, the reaction mixture was cooled to 70° C., and 2.5 g of (−)-narwedine seeds were added to the solution. The resulting suspension was cooled to 40° C. over 100 minutes, and stirred at this temperature for 18 hours. Thereafter, the suspension was cooled to 0° C. over 4 hours, and stirred at this temperature for an additional hour. The suspension was then filtered using a Büchner funnel to yield 45.0 g of (−)-narwedine (Yield: 85.7%; HPLC (method 7) purity 99.7%. Enantiomeric excess by HPLC (method 8, chiral) higher than 97.8%). The XRD (29) for Example 36 is as follows and is illustrated in FIG. 6:

| 2θ | Intensity |
|---|---|
| 10.542 | 8131.643 |
| 11.796 | 2014.976 |
| 11.880 | 1133.000 |
| 14.050 | 231.147 |
| 18.223 | 3738.977 |
| 18.396 | 2406.797 |
| 19.363 | 402.453 |
| 19.776 | 765.883 |
| 20.048 | 294.061 |
| 20.671 | 900.626 |
| 21.200 | 1801.000 |
| 21.305 | 2726.595 |
| 21.870 | 1127.739 |
| 23.091 | 313.326 |
| 23.524 | 209.745 |
| 25.288 | 1770.595 |
| 26.945 | 374.648 |
| 28.574 | 639.902 |
| 29.143 | 526.515 |
| 29.760 | 368.209 |
| 30.236 | 691.233 |
| 31.622 | 347.137 |
| 33.223 | 324.497 |
| 34.596 | 463.359 |
| 34.712 | 350.300 |
| 38.923 | 338.537 |
| 41.024 | 311.937 |
| 41.213 | 533.530 |
| 49.519 | 176.272 |

### Example 37

### Preparation of (−)-Narwedine-BF₃ Complex

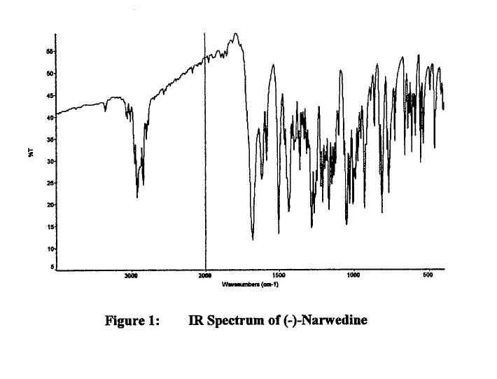

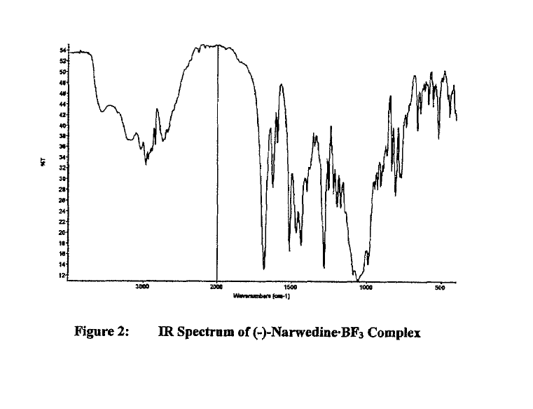

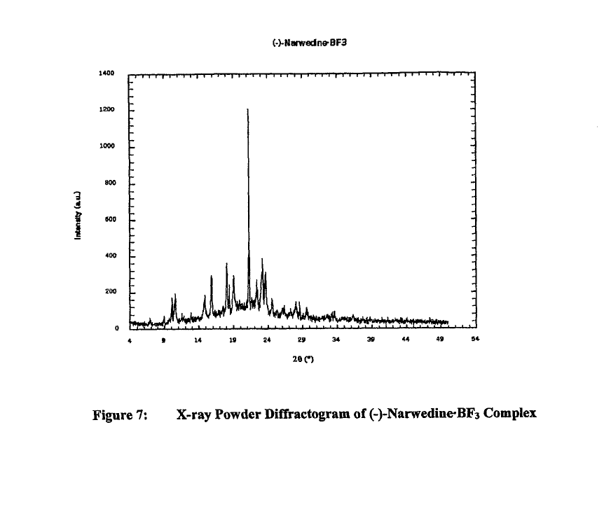

In a 10 ml round-bottomed reaction flask, 0.50 g (1.75 mmol) of (−)-narwedine were suspended in 2.5 mL of THF at room temperature. Then, 0.19 mL (1.77 mmol, 1.01 equivalents) of BF₃-THF complex was added, and the resulting suspension was stirred at room temperature for 1 hour. The solvent was then partially evaporated under reduced pressure, and diethyl ether was added to the suspension, which was then filtered at room temperature. The resulting white solid obtained was washed with diethyl ether twice to yield 039 g of (−)-narwedine-BF₃ complex (Yield: 63% yield. The IR spectra of (−)-narwedine and (−)-narwedine-BF₃ complex are illustrated in FIGS. 1 and 2 respectively. Elemental Analysis: C₁₇H₁₉NO₃.BF₃.1.25 H₂O (375.67 g/mol); C: 54.35%; H, 5.77%; N: 3.73%; F: 15.17; Found: C, 54.35%; H, 5.92%; N, 3.40%; F, 15.10. The XRD (2θ) for Example 37 is as follows and is illustrated in FIG. 7:

| 2θ | Intensity |
|---|---|
| 6.913 | 59.327 |
| 8.988 | 72.047 |
| 10.092 | 173.788 |
| 10.579 | 192.974 |
| 11.512 | 75.675 |
| 12.869 | 84.526 |
| 13.113 | 66.132 |
| 13.506 | 60.758 |
| 14.852 | 157.806 |
| 15.803 | 291.057 |
| 16.803 | 103.204 |
| 17.478 | 126.308 |
| 18.024 | 358.633 |
| 18.386 | 229.929 |
| 19.026 | 272.485 |
| 19.526 | 141.786 |
| 19.871 | 156.994 |
| 20.601 | 116.383 |
| 21.217 | 1174.555 |
| 21.594 | 165.204 |
| 22.331 | 260.984 |
| 23.148 | 377.997 |
| 23.600 | 309.646 |
| 23.851 | 139.631 |
| 24.558 | 162.546 |
| 24.880 | 85.012 |
| 25.617 | 85.769 |
| 26.265 | 122.827 |
| 27.192 | 99.554 |
| 28.008 | 132.222 |
| 28.523 | 142.673 |
| 29.530 | 104.776 |
| 33.583 | 91.962 |
| 38.271 | 59.430 |

### Example 38

### Preparation of (−)-Galanthamine.BF₃ Complex

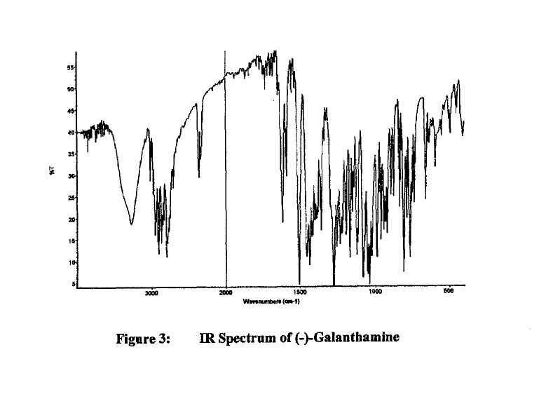

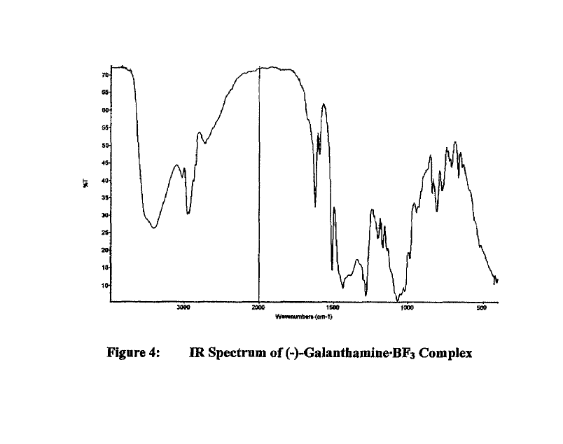

In a 25 ml round-bottomed reaction flask, 0.20 g (0.70 mmol) of (−)-narwedine was suspended in a solution of 0.075 mL (0.70 mmol, 1.0 equivalents) of BF₃-THF in 1.4 mL of THF at 0° C. The suspension was stirred at 0° C. for 30 minutes. Next, 1.4 mL (1.40 mmol, 2.0 equivalents) of L-Selectride 1.0M solution in THF was added to the reaction flask. The solution was then stirred at 0° C. for 4 hours, heated to room temperature and stirred overnight to yield a white solid. The solid was filtered and washed with diethylether to yield 0.13 g of (−)-galanthamine-BF₃ (Yield: 65%). The Ht spectra of (−)-galanthamine and (−)-galanthamine.BF₃ complex are illustrated in FIGS. 3 and 4 respectively.

### Example 39

### Preparation of (−)-Galanthamine Hydrobromide

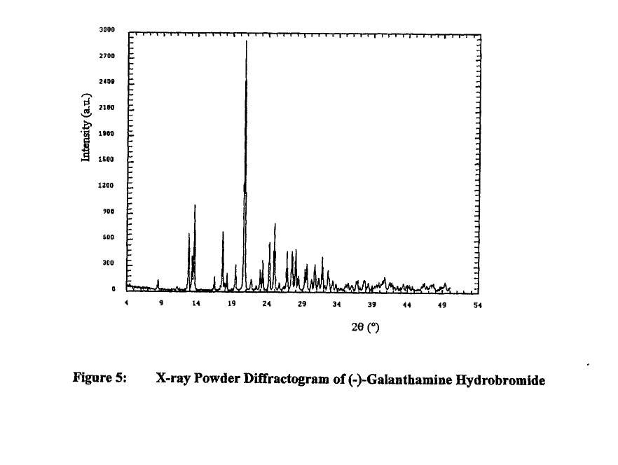

In a three necked 250 ml round-bottomed reaction flask, 5.0 g (17.52 mmol) of (−)-narwedine was suspended in a solution of 1.93 mL (17.52 mmol, 1.0 equivalents) of BF₃-THF in 25 mL of THF at 0° C. The suspension was stirred at 0° C. for 35 minutes after which 44 mL (44 mmol, 2.5 equivalents) of L-Selectride 1.0 M solution in THF was added to the reaction flask. The solution was then stirred at 0° C. for 6 hours. Next, 10 mL of THF were added to the reaction followed by 25 mL of methanol. The solvents were then distilled under vacuum, and the residue was dissolved in a mixture of 45 mL of ethyl acetate, 5 mL of THF and 20 mL of 0.1 M NaOH solution. The organic phase was then extracted and evaporated under vacuum. Next, the residue was dissolved in 45 mL of isopropanol, and the solution was filtered, cooled to 0° C. Thereafter, 42 mL (36.8 mmol, 2.1 equivalents) of 48% HBr solution in water was added to the solution to yield 5.26 g of (−)-galanthamine hydrobromide (Yield: 82% yield), which was isolated by filtration. The powder X-Ray diffraction pattern of (−)-galanthamine hydrobromide is illustrated in FIG. 5.

The obtained (−)-galanthamine hydrobromide has a powder X-Ray diffraction pattern substantially identical to the powder X-Ray diffraction pattern derived from the crystal structure published in Acta Cryst., C53, 128-1286 (1997).

Analysis of the obtained (−)-galanthamine hydrobromide: HPLC (method 1): 98.69% galanthamine, 0.41% lycoramine, 0.04% epigalanthamine, 0.36% narwedine, 0.50% other impurities. After crystallization in an isopropanol-48% HBr 9:1 mixture: 99.39% galanthamine, 0.18% lycoramine, 0.04% epigalanthamine, 0.08% narwedine, 0.31% other impurities; HPLC (method 2, chiral): less than 0.1% (+)-galanthamine. After crystallization in an isopropanol-48% HBr 9:1 mixture: (+)-galanthamine not detected.

### Example 40

### Preparation of (−)-Galanthamine Hydrobromide

In this Example, all the solutions were bubbled with N₂ and cooled to 0° C. prior to use. In a 2 L, round-bottomed cylindrical jacketed reactor, 78.6 g of (−)-narwedine was suspended in a solution of 38.5 g of BF₃-THF in 409 mL of THF under nitrogen atmosphere. The suspension was then cooled to −20° C., and 689 mL of L-Selectride 1.0 M solution in THF was added to the suspension. The resulting solution was then heated to 0° C. over 3 hours, and 79 mL of deionized water was added to the solution, followed by the addition of 705 mL of ethanol and 79 mL of 48% HBr in water. The resulting suspension was then filtered using a Büchner funnel, under air atmosphere, and washed with 79 mL of ethanol to produce a quantitative yield of (−)-galanthamine hydrobromide (HPLC (method 7) result: 98.31% galanthamine, 0.56% dehydroxygalanthamine).

### Example 41

### Preparation of (−)-Galanthamine Hydrobromide, Substantially Free of Dehydroxygalanthamine Fall Steps Under Inert Atmosphere)

In this Example, all the solutions were bubbled with N₂ for 15 minutes and cooled to 0° C. prior to use. In a 500 mL, round-bottomed cylindrical jacketed reactor, 20.0 g of (−)-narwedine was suspended in a solution of 9.89 g of BF₃-THF in 100 mL of THF under nitrogen atmosphere. The suspension was then cooled to −20° C., and 176 mL of L-Selectride 1.0 M solution in THF was added to the suspension. The resulting solution was then heated to 0° C. over 3 hours, and 20 mL of deionized water was added to the solution, followed by the addition of 180 mL of ethanol and 20 mL of 48% HBr in water. The resulting suspension was filtered under N₂, and washed with 40 mL of ethanol to produce a quantitative yield of (−)-galanthamine hydrobromide (HPLC (method 7) result: 99.58% galanthamine, 0.02% dehydroxygalanthamine).

### Examples 42-44

Examples 42-44 illustrate the effect of the pH during the quenching of the excess of L-selectride once the reduction of (−)-narwedine to (−)-galanthamine has been completed.

| | | % Dehydroxy- | % Dehydroxy- |
|---|---|---|---|
| | | galanthamine | galanthamine |
| | | before quenching | after quenching and |
| | Quenching agent | by HPLC | filtration under N₂ |
| Example | for L-selectride | (method 7) | by HPLC (method 7) |
| 42 | Aqueous HBr | 0.027% | 0.153% |
| 43 | Water | 0.024% | 0.018% |

Examples 42 and 44 illustrate the effect of the deoxygenation by N₂ bubbling of the solutions employed in the reduction of (−)-narwedine to (−)-galanthamine before their addition to the reactor.

| | Bubbling with N₂ of all the | % Dehydroxygalanthamine |
|---|---|---|
| | solutions employed before | before quenching |
| Example | their addition to the reactor | by HPLC (method 7) |
| 42 | Yes | 0.027% |
| 44 | No | 0.167% |

### Example 45

### Purification of (−)-Galanthamine Hydrobromide by Recrystallization in Water in the Presence of Ammonium Bromide

A suspension of 107.6 g of (−)-galanthamine hydrobromide (Purity by HPLC method 7, 98.31%; 0.56% dehydroxygalanthamine) and 26.9 g of ammonium bromide in 300 mL of water was heated to reflux temperature over 15 minutes, and cooled to 0° C. over one hour with stirring. After stirring for one hour at this temperature and further filtration of the resulting suspension, (−)-galanthamine hydrobromide (Purity by HPLC method 7, 99.12%; 0.20% dehydroxygalanthamine) was isolated (Yield: 94%). Two additional crystallizations yielded (−)-galanthamine hydrobromide with an HPLC purity (method 7) 99.54% (lycoramine 0.06%, epigalanthamine 0.05%, narwedine 0.07%, dehydroxygalanthamine 0.13%). HPLC (method 2, chiral) result: 100% (−)-galanthamine.

The (−)-galanthamine hydrobromide produced had the following particle size distribution (Malvern): D₁₀(v)= 10.55 μm; D₅₀(v)= 83.13 μm; D₉₀(v)= 170.96 μm. The (−)-galanthamine hydrobromide having this particle size distribution was further milled to obtain galanthamine hydrobromide with the following particle size distribution: D₁₀(v)=approximately 2 μm, D₅₀(v)= approximately 15 μm, D₉₀(v)= approximately 50 μm.

Residual Solvents in (−)-Galanthamine Hydrobromide after Drying at 60° C. Under Vacuum Until Constant Weight (6 Hours):

| | Crystallization in an | |
|---|---|---|
| | ethanol-THF mixture | |
| | (Loss on drying: | Recrystallization in water in |
| Residual solvent | 0.29% at 80° C.) | the presence of NH₄Br |
| Ethanol | 4968 ppm | Less than 100 ppm |
| Tetrahydrofuran | 5810 ppm | Less than 50 ppm |

What is claimed is:

1. A process for preparing 1-bromo-12-oxo-narwedine (Compound 5) comprising treating 2-bromo-5-hydroxy-N-[2-(4-hydroxyphenyl)ethyl]-4-methoxy-N-methylbenzamide (Compound 4) with potassium hexacyanoferrate (III) as an oxidant in conjunction with less than approximately 10 molar equivalents of at least one base.

2. The process of claim 1, wherein said at least one base comprises less than approximately 8 molar equivalents.

3. The process of claim 2, wherein said at least one base comprises approximately 5 molar equivalents.

4. The process of claim 1, wherein said at least one base is at least one of an inorganic base, an organic base, NH₃ and combinations thereof.

5. The process of claim 4, wherein said at least one base is potassium bicarbonate.

6. The process of claim 1, wherein said treating of said Compound 4 is performed at a temperature between approximately 0° C. and approximately 50° C.

7. The process of claim 6, wherein said temperature is between approximately 15° C. and approximately 35° C.

8. The process of claim 6, wherein said temperature is between approximately 20° C. and approximately 25° C.

9. The process of claim 1, further comprising a step of reducing Compound 5 to 1-bromo-4a,5,9,10-tetrahydro-6-hydroxy-3-methoxy-11-methyl-6H-benzofuro[3a,3,2-ef][2]-benzazepin-12(9H)-one (Compound 6).

10. The process of claim 9, wherein said step of reducing Compound 5 to Compound 6 comprises using a borohydride salt.

11. The process of claim 10, further comprising the use of at least one Lewis acid.

12. The process of claim 11, wherein said at least one Lewis acid is at least one of cerium (III) chloride, calcium chloride and combinations thereof.

13. 1-bromo-4a,5,9,10-tetrahydro-6-hydroxy-3-methoxy-11-methyl-6H-benzofuro[3a,3,2-ef][2]-benzazepin-12(9H)-one (Compound 6).

14. 1-bromo-12-oxo-lycoramine.

15. The compound of claim 13, wherein said Compound 6 is substantially free of 1-bromo-12-oxo-lycoramine.

16. The process of claim 1, further comprising a step of reducing Compound 6 to Compound 7.

17. (canceled)

18. The process of claim 1, further comprising a step of oxidizing Compound 7 to narwedine.

19. The process of claim 18, wherein said step of oxidizing comprises using Swern oxidation conditions.

20. The process of claim 18, wherein said step of oxidizing comprises using Oppenauer oxidation conditions.

21. The process of claim 20, wherein said Oppenauer oxidation is performed in the presence of at least one of acetone, cyclohexanone and combinations thereof.

22. (canceled)

23. The process of claim 1, further comprising a step of converting narwedine to (−)-narwedine-BF₃ comprising:
converting narwedine to (−)-narwedine; and
treating said (−)-narwedine with BF₃.THF complex.

24. (−)-Narwedine.BF₃.

25. The (−)-Narwedine.BF₃ of claim 24 further characterized by an X-ray powder diffraction pattern (29) (±0.2°) having peaks at approximately 6.913°8.988°, 10.092°, 10.579°, 11.512°, 12.869°, 13.113°, 13.506°, 14.852°, 15.803°, 16.803°, 17.478°, 18.024°, 18.386°, 19.026°, 19.526°, 19.871°, 20.601°, 21.217°, 21.594°, 22.331°, 23.148°, 23.600°, 23.851°, 24.558°, 24.880°, 25.617°, 26.265°, 27.192°, 28.008°, 28.523°, 29.530°, 33.583° and 38.271°.

26. The process of claim 1, further comprising a step of converting (−)-narwedine to (−)-galanthamine-BF₃ comprising
treating (−)-narwedine with BF₃.THF complex to produce (−)-narwedine.BF₃; and
treating the (−)-narwedine.BF₃ with L-Selectride to produce (−)-galanthamine.BF₃.

27. The process of claim 1, further comprising a step of converting (−)-narwedine to (−)-galanthamine hydrobromide comprising
treating (−)-narwedine with BF₃.THF complex to produce (−)-narwedine.BF₃;
treating the (−)-narwedine.BF₃ with L-Selectride to produce (−)-galanthamine.BF₃; and
treating the (−)-galanthamine.BF₃ with HBr to produce (−)-galanthamine hydrobromide.

28. The process of claim 1, further comprising a step of for purifying (−)-galanthamine hydrobromide comprising crystallizing (−)-galanthamine hydrobromide in water in the presence of ammonium bromide.

29. (−)-Galanthamine.BF₃.

30-36. (canceled)

37. A process for preparing dehydroxygalanthamine comprising hydrolyzing tri-sec-butylborate in the presence of (−)-galanthamine.BF₃.

38. (canceled)

39. The process of claim 37 further comprising quenching an excess of L-Selectride under acidic conditions.

40-43. (canceled)

44. The (−)-Galanthamine-BF₃ of claim 29, wherein said (−)-galanthamine.BF₃ has less than 0.2% by HPLC area of each of epigalanthamine, 1-bromo-12-oxo-lycoramine, (+)-galanthamine and dehydroxygalanthamine.

45. The (−)-Narwedine.BF₃ of claim 24 and racemic narwedine having less than 0.2% by HPLC area of lycoraminone.

46. A process for preparing epigalanthamine comprising epirimizing galanthamine under acidic conditions.
